(19) United States
(12) Patent Application Publication
(10) Pub. No.: US 2026/0260759 A1
(43) Pub. Date: Sep. 3, 2026

(54) Predicting Disease Progression In Chronic Kidney Disease Patients

(71) Applicant: BAYER AKTIENGESELLSCHAFT, Leverkusen (DE)

(72) Inventors: Ghaith SANKARI, Leverkusen (DE); Abhishek SINGH, Leverkusen (DE); Cristiana Maria RODRIGUES DE AZEVEDO VON STOSCH, Leverkusen (DE); Daniele SACCO, Leverkusen (DE)

(21) Appl. No.: 19/162,921

(22) PCT Filed: Mar. 4, 2024
(86) PCT No.: PCT/EP2024/055529

§ 371 Date: Sep. 5, 2025

(30) Foreign Application Priority Data
Mar. 10, 2023 (EP) 23161222.7
Jan. 11, 2024 (EP) 24151382.9

(51) Int. Cl. G16H 50/30; G16H 50/20

(52) U.S. Cl. CPC G16H 50/30; G16H 50/20

(57) ABSTRACT

Systems, methods, and computer programs disclosed herein relate to the prediction of disease progression in patients suffering from chronic kidney disease (CKD).

### FIELD OF THE DISCLOSURE

Systems, methods, and computer programs disclosed herein relate to the prediction of disease progression in patients suffering from chronic kidney disease (CKD).

### BACKGROUND

Kidneys play a vital role in maintaining fluid homeostasis and sodium homeostasis by filtering the blood (exocrine role). In addition, they have also an important endocrine function as they secrete various hormones and humoral factors that help regulating blood pressure, calcium bioavailability, bone formation and hemoglobin synthesis.

Chronic kidney disease (CKD) is defined as abnormalities of kidney structure or function with detrimental implications for health. CKD affects a significant part of the population worldwide and usually involves a gradual loss of kidney function. While being asymptomatic in a large proportion of patients, it is associated with an augmented risk of death, need for kidney replacement (dialysis or transplant) and cardiovascular (CV) events.

The most frequent causes of CKD include diabetes, hypertension, glomerulonephritis, and polycystic kidney disease. The main risk factors beyond diabetes and hypertension include obesity, heart disease, a family history of CKD, inherited kidney disorders, previous kidney damages and advanced age. Diagnosis is made by blood tests to measure kidney function through the assessment of estimated glomerular filtration rate (eGFR), and urine tests to measure kidney damages through the assessment of albumin and/or protein in the urine (e.g., urine albumin-creatinine ratio, uACR). Further examination, biomarker tests, imaging or kidney biopsy may be performed to determine the underlying cause.

Initially, CKD patients have no major symptoms, but at later stages, swelling in the legs, fatigue, vomiting, loss of appetite, and confusion may occur. Complications that may be related to hormonal dysfunction of the kidneys include hypertension, bone disease, and anemia. Beyond these complications which are generally associated with advanced chronic kidney disease, CKD patients are at a significantly increased risk of CV complications and/or needing kidney replacement with increased risk of death and hospitalization, even at early stages of the disease.

The objectives of the CKD management are to prevent cardiovascular and renal complications as well as to mitigate the impact of the disease on patient's quality of life. As the treatment and monitoring depends on disease severity, several staging systems based on CKD severity have been proposed. “Kidney Disease: Improving Global Outcomes”, in short KDIGO, is an independent nonprofit organization whose mission is to improve the care of patients with kidney disease worldwide. The KDIGO staging system is generally considered as a reference and is used by various medical societies and foundations including the National Kidney Foundation (NKF) in the United States and the European Renal Association (ERA). It divides CKD into six stages (1, 2, 3a, 3b, 4 and 5), depending on kidney function impairment, and 3 stages depending on kidney damage (A1, A2 and A3). Thus, each eGFR stage can be associated with one of 3 uACR-based stages.

This classification helps physicians (specialists or general practitioners/primary care physicians) and allied healthcare professionals to provide state of the art care, as each stage calls for individualized approaches to monitoring and treatment, the appropriateness of which is determined by the risk level of the patient. The six functional stages of CKD are determined using the glomerular filtration rate (GFR). The gold standard for measuring GFR is using plasma or urinary clearance of an exogenous filtration marker. However, this is a complex procedure and is generally not routinely performed. Therefore, GFR is usually estimated from the patient's serum creatinine and/or cystatin C level, in combination with demographic factors such as age, race, and gender using an estimating equation (eGFR: estimated GFR). Several equations are available to estimate GFR, the MDRD and CKD-EPI being the most frequently used. The 3 stages of kidney damage (A1, A2, A3) are assessed using the uACR equation.

US2022/0093261A1 discloses a CKD machine learning prediction system which is configured to provide a projection as to whether a patient may progress to a next stage of CKD and/or whether the patient may need to urgently start dialysis.

While useful, the CKD staging system is insufficient to determine the overall risk of a patient to experience various CV events or end-stage renal disease (ESRD), also known as kidney failure. Other factors, such as patient demographics, medical history (including previous cardiovascular diseases), prescribed drugs, biology, lifestyle, as well as the stability, progression and/or regression of CKD may determine the need for specific monitoring and treatment.

The presence and complexity of described parameters in clinical practice has led to the development of simple equations such as the kidney failure equation and computer-assisted risk scores, with the objective to predict the risk of ESRD. However, the existing scores are limited, as they do not also predict the risk of cardiovascular outcomes, including major adverse cardiovascular events (MACE). Furthermore, they only predict a level of risk, without determining the most important factors to be mitigated in order to reduce the risk of ESRD, MACE, or both. A risk score that would help to predict both the overall risk of CV events and ESRD, together with determining the most important underlying risk factors for the specific patient, would help to guide treatment decisions for the physician, and hence, would support providing the best treatment for the patient (precision medicine). The personalized nature of such a risk score would potentially also further increase patient engagement, supporting positive behavior change and treatment adherence, and as a result, clinical outcomes.

### SUMMARY

The subject matter of the independent claims of the present disclosure defines means for predicting the course of the disease of a patient suffering from CKD. Preferred embodiments are defined in the dependent claims, the description, and the drawings.

In a first aspect, the present disclosure pertains to a method implemented through a computer system that aims to define the likelihood of chronic kidney disease (CKD) progression and predict the risks associated with outcomes such as death, major adverse cardiovascular events (MACE), and the need for kidney replacement (end-stage renal disease or ESRD) in a patient. The prediction method comprises:
receiving patient data, wherein the patient data characterizes a patient suffering from chronic kidney disease (CKD) and being in one of a number of CKD stages of increasing severity, the number of CKD stages comprising: a cardiovascular event and end-stage renal disease,
generating a graph based on the patient data, the graph comprising a patient vertex, a number of stage vertices, and a number of outcome vertices,
   wherein the patient vertex represents the patient,
   wherein each stage vertex represents one of the numbers of CKD stages,
   wherein each outcome vertex represents one outcome of chronic kidney disease within a time period,
   wherein the patient vertex is connected via an edge to the stage vertex representing the CKD stage the patient is in,
   wherein each stage vertex is connected via edges to all stage vertices representing a CKD stage of greater severity, and
   wherein each stage vertex is connected to each of the outcome stages via an edge,
inputting the graph into a trained graph convolutional neural network, wherein the graph convolutional neural network was trained on training data to classify reference patients into the CKD outcomes, wherein the training data comprised, for each reference patient of a plurality of reference patients, i) a reference graph representing the reference patient and his/her chronic kidney disease course as input data, and ii) a CKD outcome of the reference patient as target data,
receiving an output from the trained graph convolutional neural network model, wherein the output indicates for one or more of the CKD outcomes a probability that CKD will reach the outcome in the patient within the time period,
outputting and/or storing the output and/or information derived from the output, and/or transmitting the output and/or information derived from the output to a separate computer system.

In another aspect, the present disclosure provides a computer system comprising:
a processor; and
a memory storing an application program configured to perform, when executed by the processor, an operation, the operation comprising:
   receiving patient data, wherein the patient data characterizes a patient suffering from chronic kidney disease (CKD) and being in one of a number of CKD stages of increasing severity, the number of CKD stages comprising: a cardiovascular event and end-stage renal disease,
   generating a graph based on the patient data, the graph comprising a patient vertex, a number of stage vertices, and a number of outcome vertices,
      wherein the patient vertex represents the patient,
      wherein each stage vertex represents one of the numbers of CKD stages,
      wherein each outcome vertex represents one outcome of chronic kidney disease within a time period,
      wherein the patient vertex is connected via an edge to the stage vertex representing the CKD stage the patient is in,
      wherein each stage vertex is connected via edges to all stage vertices representing a CKD stage of greater severity, and
      wherein each stage vertex is connected to each of the outcome stages via an edge,
   inputting the graph into a trained graph convolutional neural network, wherein the graph convolutional neural network was trained on training data to classify reference patients into the CKD outcomes, wherein the training data comprised, for each reference patient of a plurality of reference patients, i) a reference graph representing the reference patient and his/her chronic kidney disease course as input data, and ii) a CKD outcome of the reference patient as target data,
   receiving an output from the trained graph convolutional neural network, wherein the output indicates for one or more of the CKD outcomes a probability that CKD will reach the outcome in the patient within the time period,
   outputting and/or storing the output and/or information derived from the output, and/or transmitting the output and/or information derived from the output to a separate computer system.

In another aspect, the present disclosure provides a non-transitory computer readable storage medium having stored thereon software instructions that, when executed by a processor of a computer system, cause the computer system to execute the following steps:
receiving patient data, wherein the patient data characterizes a patient suffering from chronic kidney disease (CKD) and being in one of a number of CKD stages of increasing severity, the number of CKD stages comprising: a cardiovascular event and end-stage renal disease,
generating a graph based on the patient data, the graph comprising a patient vertex, a number of stage vertices, and a number of outcome vertices,
   wherein the patient vertex represents the patient,
   wherein each stage vertex represents one of the numbers of CKD stages,
   wherein each outcome vertex represents one outcome of chronic kidney disease within a time period,
   wherein the patient vertex is connected via an edge to the stage vertex representing the CKD stage the patient is in,
   wherein each stage vertex is connected via edges to all stage vertices representing a CKD stage of greater severity, and
   wherein each stage vertex is connected to each of the outcome stages via an edge,
inputting the graph into a trained graph convolutional neural network, wherein the graph convolutional neural network was trained on training data to classify reference patients into the CKD outcomes, wherein the training data comprised, for each reference patient of a plurality of reference patients, i) a reference graph representing the reference patient and his/her chronic kidney
disease course as input data, and ii) a CKD outcome of the reference patient as target data, receiving an output from the trained graph convolutional neural network, wherein the output indicates for one or more of the CKD outcomes a probability that CKD will reach the outcome in the patient within the time period,
outputting and/or storing the output and/or information derived from the output, and/or transmitting the output and/or information derived from the output to a separate computer system.

### BRIEF DESCRIPTION OF THE DRAWINGS

FIG. 1 (a), FIG. 1 (b), FIG. 1 (c) and FIG. 1 (d) schematically show different examples of graphs.

FIG. 2 (a), FIG. 2 (b), FIG. 2 (c), and FIG. 2 (d) show examples of adjacency matrices in the form of spreadsheets.

FIG. 3 shows schematically an example of a graph convolutional neural network.

FIG. 4 shows schematically another example of a graph convolutional neural network.

FIG. 5 shows schematically an embodiment of the computer-implemented prediction method of the present disclosure in the form of a flowchart.

FIG. 6 shows schematically an embodiment of the computer-implemented training method of the present disclosure in the form of a flowchart.

FIG. 7 illustrates a computer system according to some example implementations of the present disclosure in more detail.

FIG. 8 shows an exemplary and schematic output of the computer system of the present disclosure.

FIG. 9 shows another exemplary and schematic output of the computer system of the present disclosure.

FIG. 10 explains how the graph convolutional neural network model shortlists a cohort based on likelihood.

FIG. 11 shows a receiver operating characteristic curve for the example described in the Example section.

### DETAILED DESCRIPTION

The invention will be more particularly elucidated below without distinguishing between the aspects of the invention (method, computer system, computer-readable storage medium). On the contrary, the following elucidations are intended to apply analogously to all the aspects of the invention, irrespective of in which context (method, computer system, computer-readable storage medium) they occur.

If steps are stated in an order in the present description or in the claims, this does not necessarily mean that the invention is restricted to the stated order. On the contrary, it is conceivable that the steps can also be executed in a different order or else in parallel to one another, unless one step builds upon another step, this absolutely requiring that the building step be executed subsequently (this being, however, clear in the individual case). The stated orders are thus preferred embodiments of the present disclosure.

As used herein, the articles “a” and “an” are intended to include one or more items and may be used interchangeably with “one or more” and “at least one.” As used in the specification and the claims, the singular form of “a”, “an”, and “the” include plural referents, unless the context clearly dictates otherwise. Where only one item is intended, the term “one” or similar language is used. Also, as used herein, the terms “has”, “have”, “having”, or the like are intended to be open-ended terms. Further, the phrase “based on” is intended to mean “based at least partially on” unless explicitly stated otherwise. Further, the phrase “based on” may mean “in response to” and be indicative of a condition for automatically triggering a specified operation of an electronic device (e.g., a controller, a processor, a computing device, etc.) as appropriately referred to herein.

Some implementations of the present disclosure will be described more fully hereinafter with reference to the accompanying drawings, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art.

The present disclosure provides means to predict the disease course of a CKD patient, including the risk of occurrence of adverse events.

The prediction is done using a trained machine learning model. Such a “machine learning model”, as used herein, may be understood as a computer implemented data processing architecture. The machine learning model can receive input data and provide output data based on that input data and on parameters of the machine learning model (model parameters). The machine learning model can learn a relation between input data and output data through training. In training, parameters of the machine learning model may be adjusted in order to provide a desired output for a given input.

The process of training a machine learning model involves providing a machine learning algorithm (that is the learning algorithm) with training data to learn from. The term “trained machine learning model” refers to the model artifact that is created by the training process. The training data must contain the correct answer, which is referred to as the target. The learning algorithm finds patterns in the training data that map input data to the target, and it outputs a trained machine learning model that captures these patterns.

In the training process, input data is inputted into the machine learning model and the machine learning model generates an output. The output is compared with the (known) target. Parameters of the machine learning model are modified in order to reduce the deviations between the output and the (known) target to a (defined) minimum.

A loss function can be used to quantify the deviations between the output and the target. If, for example, the output and the target are numbers, the loss function could be the difference between these numbers. In this case, a high absolute value of the loss function can mean that a parameter of the model needs to undergo a strong change.

In the case of vector-valued outputs, for example, difference metrics between vectors such as the root mean square error, a cosine distance, a norm of the difference vector such as a Euclidean distance, a Chebyshev distance, an Lp-norm of a difference vector, a weighted norm or any other type of difference metric of two vectors can be chosen. These two vectors may for example be the desired output (target) and the actual output.

In the case of higher dimensional outputs, such as two-dimensional, three-dimensional or higher-dimensional outputs, for example an element-wise difference metric can be used. Alternatively or additionally, the output data may be transformed, for example to a one-dimensional vector, before computing a loss.

The modification of model parameters and the reduction of the loss can be done in an optimization procedure, for example in a gradient descent procedure.

The training data used to train the machine learning model of the present disclosure comprises, for each reference patient of a multitude of reference patients i) a reference graph as input data and ii) CKD outcome data as target data.

The term “multitude” as it is used herein means an integer greater than 1, usually greater than 10, preferably greater than 100.

The term “reference” is used in this disclosure to distinguish the data used to train and/or validate the machine learning model from the data used to make predictions using the trained model. Thus, the data used to train and/or validate the machine learning model represent “reference patients” whereas data used for prediction purposes represent a new patient (i.e., a patient which is not a reference patient). A “reference graph” represents a reference patient and his/her chronic kidney disease course, i.e., the reference patient's CKD progression within a defined time period. The term “(reference) patients” means “reference patients or patients”. However, the term “reference” is not to be understood in any other restrictive sense; this distinction serves only to prevent a clarity objection in patent grant proceedings. If at any point in this disclosure a statement is made in relation to a “graph”, it generally also applies to a “reference graph”, even if the statement does not always refer to the graph as a “(reference) graph”. Sometimes the addition “(reference)” has been omitted for reasons of better readability.

In discrete mathematics, and in graph theory in particular, a “graph” is a structure consisting of a set of entities where some pairs of entities are “related” in some sense. D. Ahmedt-Aristizabal et al. disclose graph-based deep learning for medical diagnosis and analysis: D. Ahmedt-Aristizabal et al.: Graph-based Deep learning for Medical Diagnosis and Analysis: Past, Present and Future, 2021, arXiv:2105.13137v1.

In the present case, a (reference) graph is used as a representation of a (reference) patient and his/her (real or potential) chronic kidney disease course.

A graph comprises a number of vertices (also referred to as nodes) and edges (also referred to as links) between vertices. Each vertex represents an entity, and each edge represents a relation between a pair of entities.

In the present case, a (reference) graph comprises a (reference) patient vertex, a number of stage vertices, and a number of outcome vertices.

The (reference) patient vertex represents the (reference) patient.

Each stage vertex represents one of a number of CKD stages. For example, if the training and prediction is based on the KDIGO staging system, there can be six stage vertices, each stage vertex representing one of the KDIGO stages 1, 2, 3a, 3b, 4, and 5. It is also possible that, in addition to stages based on the (estimated) glomerular filtration rate (GFR, eGFR), stages based on urinary albumin-creatinine ratio (uACR) may be considered. For example, each of the (e) GFR stages can be linked to each of the uACR stages, resulting in a number of 15 CKD stages. In principle, of course, it is also possible to consider only CKD stages determined on the basis of the albumin-creatinine ratio in the urine. Preferably, however, CKD stages determined at least partially on the basis of the (estimated) glomerular filtration rate of (referenced) patients are used.

It should be noted that the present invention is not limited to any particular CKD staging system and/or number of CKD stages. The CKD staging system of the KDIGO is described in this disclosure as an example of a possible CKD staging system; however, the invention is suitable for all conceivable CKD staging systems and numbers of CKD stages. For example, the UK Kidney Association published a CKD staging system comprising five stages (G1, G2, G3, G4, G5) based on eGFR, and three stages (A1, A2, A3) based on uACR. The invention can therefore also be carried out on the basis of the CKD staging system of the UK Kidney Association. However, it should be ensured that the data used for training and subsequent prediction are based on the same or an equivalent CKD staging system.

Similarly, the present invention is not limited to a specific manner in which the parameters for determining the CKD stages are determined. For example, if CKD stages are based on an estimated glomerular filtration rate, the equation used to determine the estimated glomerular filtration rate is irrelevant to the performance of the present invention; it can be any equation which is able to estimate a glomerular filtration rate of a patient.

Likewise, it is not mandatory that the amount of albumin in the urine is related to the amount of creatine. It is conceivable to use an albumin amount (albumin level) that uses a different reference quantity.

Finally, the present invention is open to future CKD stages that may be determined based on parameters other than (e) GFR and/or uACR and/or in which additional/other parameters are considered.

Each outcome vertex represents one outcome of a number of CKD outcomes within a certain time period. The number of CKD outcomes can be 1, 2, 3, 4, 5, 6, 7, 8, 9 or 10 or any other number. The time period may begin, for example, when a (reference) patient is diagnosed with CKD (baseline CKD stage) and/or when the CKD stage the patient is in is determined and/or at the time the prediction is made, and may last, for example, 6 months or 1 year or 18 months or 2 years or 30 months or 3 years or 4 years or 5 years or 6 years or 7 years or 8 years or 9 years or 10 years or some other time period. The time period can also be the patient's remaining lifetime.

In a preferred embodiment of the present disclosure, the time period lasts 2 years. In another preferred embodiment of the present disclosure, the time period lasts 3 years. In another preferred embodiment of the present disclosure, the time period lasts 4 years. In another preferred embodiment of the present disclosure, the time period lasts 5 years.

Examples of CKD outcomes within the time period are: end-stage renal disease (ESRD), one or more specific major adverse cardiovascular events (MACE) and/or generally any CV event, and death. ESRD relates to the need for kidney replacement (dialysis or transplant). “Cardiovascular (CV) event” refers to any CV incident that may influence health. MACE include, but are not restricted to, myocardial infarction, unstable angina, need for revascularization, heart failure and any type of stroke.

A CKD outcome can also be “remaining in one CKD stage” or “moving to a higher CKD stage” (i.e., a CKD stage with a higher severity).

In an embodiment, the following four CKD outcomes are used: CV event, ESRD, death, no outcome. Here, “CV event” means that some CV event, including major adverse CV event, occurs within the time period. “No outcome” means that none of the other CKD outcomes (CV event, ESRD, death) occurs within the time period.

In another embodiment, the CKD outcomes include: CV event, ESRD. Here, “CV event” means that some CV event, including major adverse CV event, occurs within the time period.

The (reference) patient vertex is connected via a directed edge to the stage vertex representing the CKD stage the (reference) patient is in.

The term “directed edge” refers to the concept of “message passing” as described, for example, in the following article: J. Gilmer et al.: Neural Message Passing for Quantum Chemistry, Proceedings of the 34^{th} International Conference on Machine Learning, 2017, Vol 70, pp. 1263-1272. It means that two vertices connected with a directed edge cannot influence each other and cannot exchange information with each other, but that only one of the vertices can influence the other and information can flow only from one to the other. The direction of influence and information flow is often indicated by an arrow in the graph. In contrast, vertices connected by an undirected edge influence each other and exchange information with each other.

Further connections between the vertices of the graph are based on whether the patient is a reference patient or a new patient. For reference patients, the course of the disease within the defined time period is known. The connections between the vertices are set so that the reference graph reflects the disease progression. For a new patient, the course of the disease within the defined time period is unknown (which is why it should be predicted). The graph reflects different potential disease courses. The machine learning model is trained to recognize patterns in disease courses and patient data using reference graphs from reference patients. Once trained, the machine learning model can be used to output a probability value for one or more of the possible disease courses/CKD outcomes for a new patient.

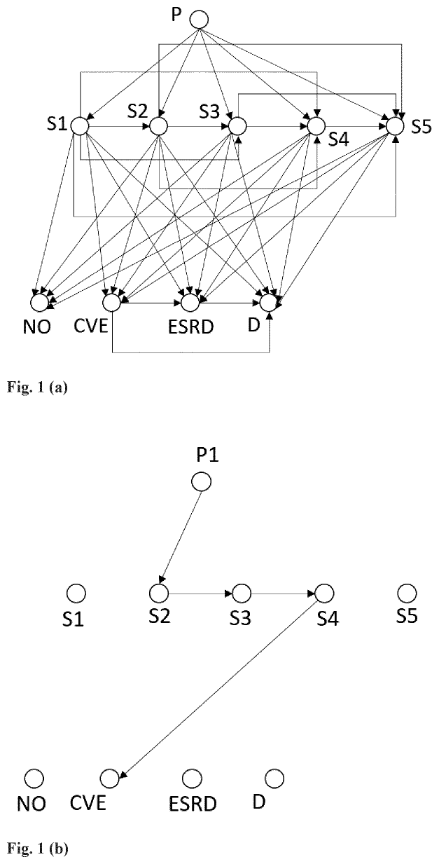

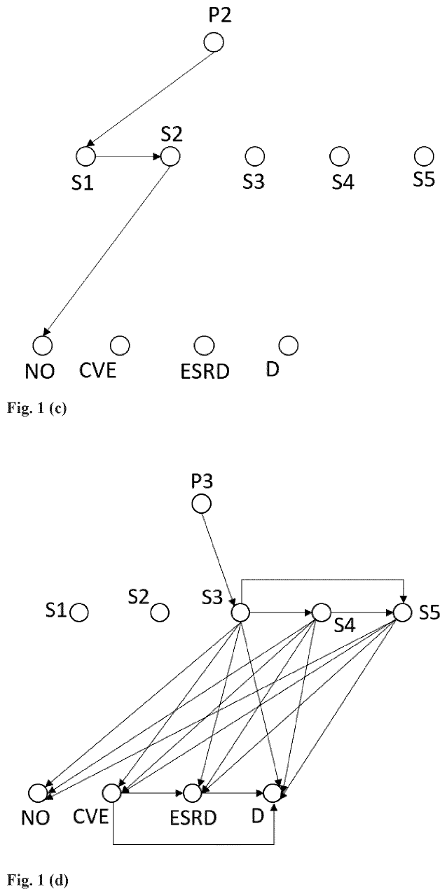

FIG. 1 (a), FIG. 1 (b), FIG. 1 (c) and FIG. 1 (d) schematically show different examples of graphs.

Each graph comprises a vertex representing a patient: vertex P in case of FIG. 1 (a), vertex P1 in case of FIG. 1 (b), vertex P2 in case of FIG. 1 (c), and vertex P3 in case of FIG. 1 (d).

Each graph further comprises five vertices S1, S2, S3, S4, and S5, each of them representing a CKD stage. Vertex S1 represents CKD stage 1, vertex S2 represents CKD stage 2, vertex S3 represents CKD stage 3, vertex S4 represents CKD stage 4, and vertex S5 represents CKD stage 5. The numbering of CKD stages is analogous to their severity, i.e., CKD stage S2 has a higher severity than CKD stage S1, CKD stage S3 has a higher severity than CKD stage S2, CKD stage S4 has a higher severity than CKD stage S3, and CKD stage S5 has a higher severity than CKD stage S4.

Each graph further comprises four outcome vertices. Vertex NO represents the outcome stage “no outcome”, vertex CVE represents the outcome stage “CV (cardiovascular) event”, vertex ESRD represents the outcome stage “end-stage renal disease (ESRD)”, and vertex D represents the outcome “death”, as described above. Each outcome refers to a CKD outcome within the defined period of time.

The outcomes CVE, ESRD, and D represent the highest-risk health situations that a patient suffering from CKD would likely want to avoid. Mitigating their related risk is one of the main objectives of CKD management. If the patient succeeds in avoiding the aforementioned situations, the outcome is “no outcome” (NO). As mentioned above, “no outcome” means that none of the other CKD outcomes (CV event, ESRD, death) occurs within the time period.

FIG. 1 (a) shows schematically and exemplarily the structure of a graph with all potential connections between the existing vertices.

The vertex P representing the patient is connected via a directed edge with each of the vertices S1, S2, S3, S4 representing a CKD stage.

Each vertex representing a CKD stage is connected via a directed edge with each vertex representing a higher CKD stage, i.e., a CKD stage of higher severity. So, S1 is connected via directed edges with S2, S3, S4, and S5; S2 is connected via directed edges with S3, S4, and S5; S3 is connected via directed edges with S4 and S5; S4 is connected via a directed edge with S5.

The outcome vertices are connected according to their severity, too: CVE is connected via a directed edge with ESRD and D; ESRD is connected via a directed edge with D.

FIG. 1 (b) shows schematically an example of a reference graph for a reference patient. Such a reference graph can be used for training the machine learning model.

The vertex P1 representing the reference patient is connected via a directed edge with the vertex S2 representing the CKD stage the reference patient was in at baseline (when CKD was diagnosed). So, the reference patient represented by vertex P1 was in CKD stage 2 of the CKD staging system. Within the defined time period, the reference patient's health deteriorated. The reference patient moved from CKD stage 2 to CKD stage 3 and then to CKD stage 4. Finally, a CV event occurred (outcome: CVE).

FIG. 1 (c) shows schematically another example of a reference graph for another reference patient. Such a reference graph can be used for training the machine learning model, too.

The vertex P2 representing the reference patient is connected via a directed edge with the vertex S1 representing the CKD stage the reference patient was in at baseline. So, the reference patient represented by vertex P2 was in CKD stage 1 of the staging system. Within the defined time period, the reference patient's health deteriorated slightly (the reference patient moved from CKD stage 1 to CKD stage 2), but then remained stable (outcome: NO).

FIG. 1 (d) schematically shows an example of a graph for a new patient for whom a prediction is to be made about the course of CKD within the defined time period. At the time the prediction is to be made, the patent is in CKD stage 3. Therefore, vertex P3, representing the new patient, is connected to vertex S3, representing CKD stage 3, via a directed edge.

From this CKD stage, the further course of the disease is still completely open. Therefore, vertex S3, representing CKD stage 3, is connected to all other vertices (S4, S5) representing a CKD stage with a higher severity via directed edges. In addition, vertex S4 is connected to vertex S5 via a directed edge. Furthermore, the vertices S3, S4, S5 representing CKD stages 3, 4, 5 are connected to the vertices NO, CVE, ESRD, D, each representing a possible outcome. The outcomes CVE, ESRD, D are connected in ascending severity via directed edges.

So, while FIG. 1 (b) and FIG. 1 (c) show reference graphs of reference patients for whom the disease progression is already known (which is why these graphs can be used for training), in the case of the graph shown in FIG. 1 (d), only the starting point is known for the new patient (which is CKD stage S3); the further progression allows many different possible paths along the directed edges.

It should be noted that FIG. 1 (a), FIG. 1 (b), FIG. 1 (c) and FIG. 1 (d) show only one example of a graph structure. Other structures of graphs are also possible that include different and/or more and/or fewer stages and/or outcomes. The edges and/or types of edges may also be different.

For each element (vertex, edge) of a graph, there is information that describes and/or characterizes the element and/or the entity/relation it represents. This information is referred to as “embedding” in this disclosure. An embedding is a numerical representation of one or more properties (features) of an entity or relation. A vertex embedding provides information about the entity the respective vertex represents; an edge embedding provides information about the relation between a pair of entities. There can be a global embedding, providing information about the whole graph.

So, an embedding can be a number, a tuple, a vector, a matrix, a tensor, or some other arrangement of numbers. Instead of the term “embedding”, the terms “feature vector” and “properties matrix” are sometimes used, although an embedding as described is not limited to a vector or a matrix.

An embedding that represents one or more characteristics of a patient (patient embedding) can be generated from patient data.

Patient data may include, e.g.: demographic data (age, sex, body size (height), body weight, body mass index, ethnicity), resting heart rate, heart rate variability and other data derived from heart rate, glucose concentration in blood and urine, body temperature, impedance (e.g., thoracic impedance), blood pressure (e.g., systolic and/or diastolic arterial peripheral blood pressure), current CKD stage, GFR, eGFR, urine albumin-to-creatinine ratio (uACR), blood measurement values (e.g., blood sugar, oxygen saturation, erythrocyte count, hemoglobin content, leukocyte count, platelet count, inflammation values, blood lipids including low-density lipoprotein cholesterol (LDL) and high-density lipoprotein cholesterol (HDL), ions including Na⁺ and corrected calcium), pre-existing cardiovascular disease(s), genetic background, lifestyle information about the life of the patient, such as consumption of alcohol, smoking, and/or exercise and/or the patient's diet, information about how long the patient has had one or more diseases (especially diabetes, hypertension, and/or cardiovascular disease), medical intervention parameters such as regular medication, occasional medication, or other previous or current medical interventions and/or other information about the patient's previous and/or current treatments and/or reported health conditions and/or combinations thereof.

Patient data may comprise information from an electronic medical record (EMR, also referred to as electronic health record (EHR)). The EMR may contain information about a hospital's or physician's practice where certain treatments were performed and/or certain tests were performed, as well as various other (meta-) information about the patient's treatments, medications, tests, and physical and/or mental health records.

Patient data may comprise information about a person's condition obtained from the person himself/herself (self-assessment data, (electronic) patient reported outcome data (e) PRO)). Besides objectively acquired anatomical, physiological, physical and/or behavioral data, the well-being of the patient also plays an important role in the monitoring of health. Subjective feeling can also make a considerable contribution to the understanding of objectively acquired data and of the correlation between various data. If, for example, it is captured by sensors that a person has experienced a physical strain, for example because the respiratory rate and the heart rate have risen, this may be because just low levels of physical exertion in everyday life place a strain on the person; however, another possibility is that the person consciously and gladly brought about the situation of physical strain, for example as part of a sporting activity. A self-assessment can provide clarity here about the causes of physiological features.

Subjective feeling can be collected by using a self-assessment unit, with which the patient can record information about subjective health status. For example, the patient may be asked to answer a list of questions. Preferably, the questions are answered with the aid of a computer system (e.g., laptop computer, tablet computer and/or smartphone). One possibility is that the patient has questions displayed on a screen and/or read out via a speaker. One possibility is that the patient inputs information into a computer by, e.g., inputting text via an input device (e.g., keyboard, mouse, touchscreen and/or a microphone (by means of speech input)). A chatbot is conceivable to facilitate the input of all items of information for the patient. It is conceivable that the questions are recurring questions which are to be answered once or more than once a day or a week by a patient. It is conceivable that some of the questions are asked in response to a defined event. It is, for example, conceivable that it is captured by means of a sensor that a physiological parameter is outside a defined range (e.g., an increased respiratory rate is established and/or the blood pressure exceeds a pre-defined threshold). As a response to this event, the patient can, for example, receive a message via his/her smartphone or smartwatch or the like that a defined event has occurred and that said patient should please answer one or more questions, for example to find out the causes and/or the accompanying circumstances in relation to the event.

Patient data can be provided by the patient and/or any other person such as a physician and/or a physician assistant. Patient data may be entered into one or more computer systems by said person or persons via input means (such as a keyboard, a touch-sensitive surface, a mouse, a microphone, and/or the like).

Patient data can be captured (e.g., automatically) by one or more sensors, e.g., blood pressure sensor, motion sensor, activity tracker, blood glucose meter, heart rate meter, thermometer, impedance sensor, microphone (e.g., for voice analysis) and/or others.

Patient data can be measured by a laboratory and stored in a data storage by laboratory personnel.

Patient data can be read from one or more data storages.

In an embodiment, patient data comprises: a glomerular filtration rate and/or an albumin level.

In another embodiment, patient data comprises: age, sex, current CKD stage, GFR or eGFR, uACR.

In another embodiment, patient data comprises: age, sex, current CKD stage, GFR or eGFR, uACR, ethnicity, body mass index, LDL, systolic blood pressure.

In another embodiment, patient data comprises: age, sex, current CKD stage, GFR or eGFR, uACR, ethnicity, body mass index, LDL, systolic blood pressure, corrected calcium, information on whether the patient smokes, information on whether the patient suffers from diabetes.

In another embodiment, patient data comprises one or more of the following: age, sex, ethnicity, glomerular filtration rate, albumin level (e.g., albumin-to-creatinine ratio in urine), body-mass index, blood pressure.

In another embodiment, patient data comprise one or more of the following: age; sex; ethnicity; current CKD stage (e.g., according to the KDIGO staging system); presence of one or more of the following conditions and/or information about how long the one or more conditions have been present: hypertension, diabetes, cardiovascular disease(s); GFR; eGFR; uACR; LDL; corrected calcium; systolic (e.g., arterial, peripheral) blood pressure; body mass index; information on whether the patient smokes and/or what quantities (e.g., in the form of an average number of cigarettes per day) the patient consumes.

In another embodiment, patient data comprises information from which the current CKD stage of the patient can be derived and/or information about the current CKD stage itself.

It should be noted that some patient data can be calculated from other patient data. For example, the body mass index can be calculated from the patient's height and weight. Instead of the body mass index, the patient's height and weight can also be used as patient data and vice versa. The same applies for the albumin-to-creatinine ratio. Depending on the CKD staging system used, this may also apply to the current CKD stage, which can be derived (calculated) from the glomerular filtration rate and albumin-to-creatinine ratio, for example. Thus, if patient data is mentioned in this disclosure from which other patient data can be calculated or which can be calculated from other patient data, the other patients are also included in the patient data mentioned.

The machine learning model of the present disclosure has been shown to be tolerant of missing information on smoking habits and corrected calcium.

Instead of or in addition to the GFR value and/or eGFR value, a slope of the corresponding value as a function of time (e.g., over a period of the last weeks and/or months) can be used.

For patient data that is in the form of numbers, these numbers can be used directly to generate an embedding (feature vector). For categorical patient data, numbers can be assigned to the different categories (e.g., the number 1 for female and the number 2 for male), or one-hot encodings can be used.

“One-hot encoding” is a method to quantify categorical data. In short, this method produces a vector with length equal to the number of categories in the data set. If a data point belongs to the i^{th} category, then components of this vector are assigned the value 0 except for the i^{th} component, which is assigned a value of 1.

The individual values of the patient data can be combined in an embedding to generate the patient embedding. The individual values can be arranged, for example, in a vector or in the form of a matrix.

The connections of the vertices in the graph via the edges can be described in terms of an adjacency matrix. An adjacency matrix of a graph is a matrix that stores which vertices of the graph are connected by an edge. It has a row and a column for each vertex, resulting in an n×n matrix for n vertices. An entry in the i^{th} row and j^{th} column indicates whether an edge leads from the i^{th} to the j^{th} vertex. If there is a 1 at this position, an edge leads from the i^{th} to the j^{th} vertex; if there is a 0, no edge leads from the i^{th} to the j^{th} vertex. If the graph is undirected (i.e., all its edges are bidirectional), the adjacency matrix is symmetric.

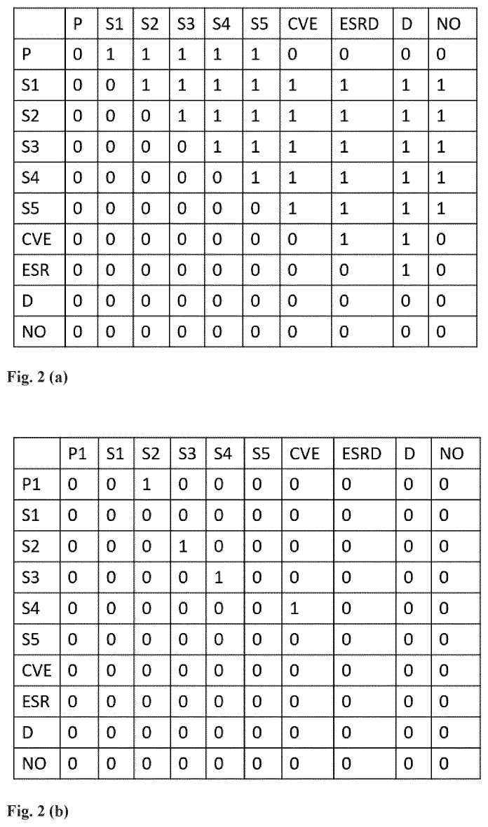

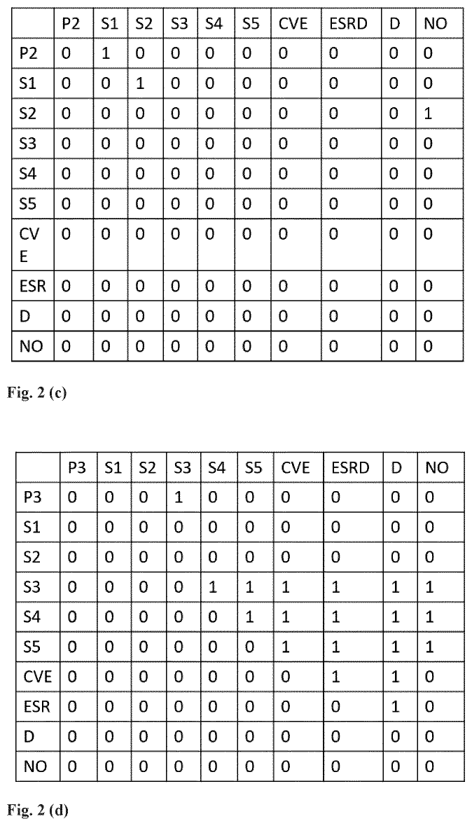

FIG. 2 (a), FIG. 2 (b), FIG. 2 (c), and FIG. 2 (d) show examples of adjacency matrices in the form of spreadsheets. FIG. 2 (a) shows the adjacency matrix for the graph shown in FIG. 1 (a); FIG. 2 (b) shows the adjacency matrix for the graph shown in FIG. 1 (b); FIG. 2 (c) shows the adjacency matrix for the graph shown in FIG. 1 (c); FIG. 2 (d) shows the adjacency matrix for the graph shown in FIG. 1 (d).

Edges can be weighted in a graph. Such weights can be taken into account in an adjacency matrix by replacing the value 1 by the respective weight.

For example, the edges between the stage vertices can be assigned a weight reflecting the percentage of reference patients who were diagnosed with the baseline CKD stage and also diagnosed with the destination CKD stage during their journey.

Similarly, the edges between stage vertices and outcome vertices can be assigned a weight reflecting the percentage of reference patients who were diagnosed with the baseline CKD stage and developed the destination outcome during their journey.

The weights can be fixed numbers and/or responsive variables that reflect the targeted population and/or variables that the machine learning model should learn as part of the training.

In the following, one possibility for the weighting is described in more detail, without wanting to limit the invention to this possibility:
Each (reference) graph consists of three types of vertices: stage vertices, outcome vertices, and patients' vertices.
Stage vertices: Each stage vertex has a properties matrix (embedding) as a placeholder for the connected vertices' properties. The values of this matrix are initially set to 0 during model training.
Outcome vertices: Each outcome vertex has a properties matrix (embedding) as a placeholder for the connected vertices' properties. The values of this matrix are initially set to 0 during model training.
Patients' vertices: Each patient vertex has a properties matrix (patient embedding) representing the medical history, biological information, and behavioral variables for the (reference) patient at that point in time. These data are aggregated to represent the (reference) patient journey.
Each edge between two vertices has a weight assigned according to the following rules:
   Patient to patient connection: There is no edge between two patients' vertices unless these vertices belong to the same patient over different CKD stages. The weight in this case is 1.
   Patient to stage connection: Having a connection between a patient vertex and a stage vertex means the (reference) patient is diagnosed with CKD at that stage. The weight in this case is 1.
   Patient to outcome connection: Having a connection between a patient vertex and an outcome vertex means the reference patient developed that outcome during their journey. The weight in this case is 1.
   Stage to stage connection: The weight represents the percentage of reference patients who were diagnosed with the source vertex stage and also diagnosed with the destination vertex stage during their journey. The weight is less than 1 and equal or larger than 0.
   Stage to outcome connection: The weight represents the percentage of reference patients who were diagnosed with the source vertex stage and developed the destination vertex outcome during their journey. The weight is less than 1 and equal or larger than 0.
Following these rules will result in a weights matrix. The final adjacency matrix is the result of the connection matrix and weights matrix.
The weights can be adjusted to the target population so that the weights can be changed for each new patient added to the cohort. It is possible that these are not trainable weights, so the model does not need to be re-trained.

A graph embedding can include global information about the (reference) graph. For example, a graph embedding can include information about the defined time period for which the prediction is to be made.

The patient embedding, stage embeddings, outcome embeddings, adjacency matrix (or any other embedding representing the connections between the vertices), and optionally a global embedding can be concatenated and/or stacked to form the (reference) graph.

Once a (reference) graph is generated for a (reference) patient, it can be inputted into the machine learning model.

Preferably, the machine learning model is or comprises a graph convolutional neural network (GCN). A graph convolutional neural network is an artificial neural network for processing data in the form of graphs.

A graph convolutional neural network usually has an input layer for inputting a graph. The input layer usually has as many input nodes as there are input values. The graph convolutional neural network usually has a number of further layers in which the inputted graph is subjected to operations. The result of the operations is a transformed graph. A prediction can then be made based on the transformed graph. Such a prediction can be a classification or a regression or another task or a combination of tasks. The prediction can be made for the graph as a whole and/or for one or more vertices and/or for one or more edges.

In the case of the present disclosure, one or more probability values for one or more outcomes are generated as output from the graph convolutional neural network.

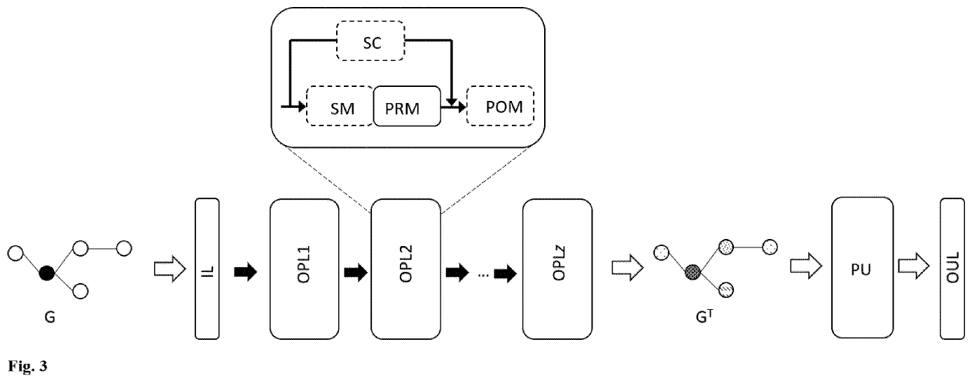

FIG. 3 shows schematically an example of a graph convolutional neural network. The graph convolutional neural network comprises an input layer IL, a number z of operation layers OPL, a prediction unit PU, and an output layer OUL.

Each operation layer OPL can comprise one or more propagation modules PRM, sampling modules SM, and/or pooling modules POM.

A propagation module PRM is used to propagate information between vertices so that the aggregated information could capture both feature and topological information. In propagation modules, convolution operators and/or recurrent operators can be used to aggregate information from neighbored vertices while skip connection operation SC can be used to gather information from former representations of vertices and mitigate over-smoothing problems.

When graphs are large, a sampling module SM can be used to conduct propagation on graphs. A sampling module SM is usually combined with a propagation module PRM.

Pooling modules POM can be used to extract information from vertices.

As shown in FIG. 3, in each operation layer OPL, a convolutional operator, recurrent operator, sampling module and/or skip connection can be used to propagate information in each layer and then the pooling module can be added to extract high-level information. These layers are usually stacked to obtain better representations.

The operation layers OPL map the graph G onto a transformed graph G^{T}.

The transformed graph G^{T} is a representation in which the information that was present in the original graph G is processed to be useful for prediction. In other words, the graph convolutional neural network is trained to process the original information contained in graph G in such a way that information is extracted, prepared, and provided (in the form of the transformed Graph G^{T}) that enables prediction.

The transformed graph G^{T} is fed to the prediction unit PU. The prediction unit can comprise several fully connected layers followed by a layer performing a non-linear activation function (e.g., a sigmoid function). The result of the prediction is provided by the output layer OUL.

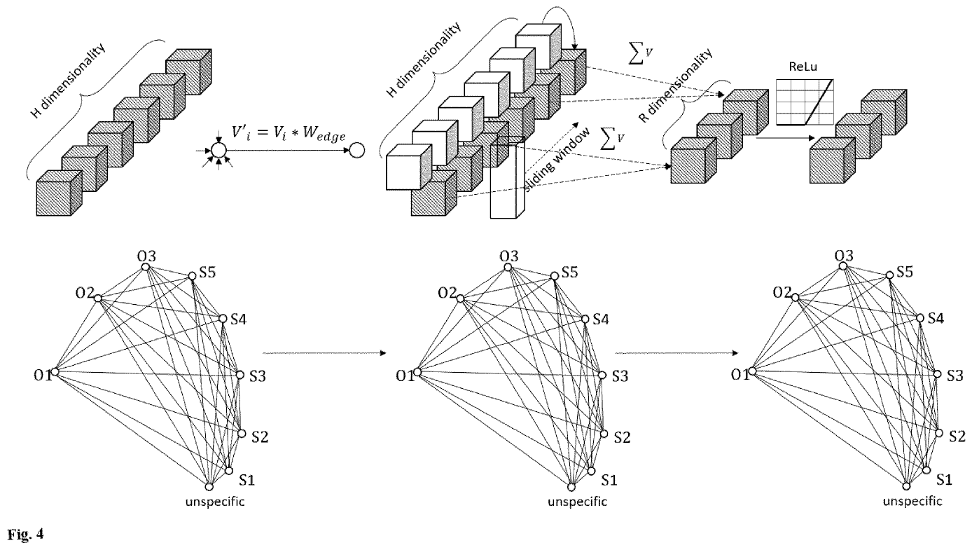

FIG. 4 shows schematically another example of a graph convolutional neural network. The graph convolutional neural network utilizes multiple convolutional layers to reduce the dimensionality of the properties matrices of vertices Vᵢ from H to R (wherein i is an index specifying the respective vertex and H and R are integers for which H>R holds.).

The convolutional layer can be either a max pooling or a sum pooling layer, and it is activated using a nonlinear activation function (such as a ReLu function). The final convolutional layer has the same dimensionality as the number of outcomes in the graph.

The output of the last convolutional layer can be fed as input to a set of fully connected (“dense”) layers, which can also be activated by a nonlinear activation function. This allows for the neural network to learn complex relationships between the input data and the target outputs.

When performing a multiclass classification task, the output layer of the graph convolutional neural network can be activated by a sigmoid function. This allows the network to classify multiple results simultaneously.

More details about graph convolutional neural networks, their architecture and operations can be found in publications on this topic (see, e.g., J. Zhou et al.: Graph neural networks: A review of methods and applications, AI Open 1, 2020, 57-81).

During training, for each reference patient of the multitude of reference patients, the reference graph representing the reference patient and his/her chronic kidney disease progression in the defined time period is inputted as input data to the machine learning model.

Each reference graph of each reference patient comprises:
a patient vertex representing the reference patient,
a first stage vertex representing a baseline CKD stage of the reference patient and a second stage vertex representing a destination CKD stage of the reference patient,
an outcome vertex representing the CKD outcome that the reference patient developed during his/her chronic kidney disease course.

It is noted that the first stage vertex and the second stage vertex can be identical, e.g., if the patient has remained in the baseline CKD stage within the time period.

It is noted that the CKD outcome that the reference patient developed during his/her chronic kidney disease course can also be “no outcome”, as described above.

The machine learning model generates output data based on the input data and model parameters. The output data is compared with the CKD outcome of the respective reference patient within the defined time period (target data).

A loss function can be used to quantify deviations between the output data and the target data. In an optimization procedure, the model parameters can be modified to reduce the deviations (the loss value calculated using the loss function). The optimization procedure can be a gradient descent procedure, for example.

During training, the architecture of the machine learning model can also be modified. For example, the number of operation layers in a graph convolutional neural network can be increased or decreased.

Training can be completed when the loss values have reached a defined minimum and/or the loss values have reached a plateau, i.e., it cannot be reduced further by modifying the model parameters.

A cross-validation method can be employed to split the data into training and validation data sets. The training data set is used in the training of the machine learning model. The validation data set is used to verify that the trained machine learning model generalizes to make good predictions.

Once the machine learning model is trained, the trained machine learning model can be used for prediction.

In prediction, a graph representing a new CKD patient is entered into the trained model as input data.

The trained machine learning model generates an output. The output indicates for one or more of the CKD outcomes defined in the training process a probability that CKD will reach the outcome in the new patient within the time period.

For example, in the case of a number m of CKD outcomes, the output may be an m-dimensional vector where each vector element indicates a probability value for one of the number m of CKD outcomes.

It is also possible for the computer system to output the most likely course of the disease.

It is also possible that the output includes several disease progression pathways, e.g., the most probable progression pathway for the patient to develop end-stage renal disease within the time period, the most probable progression pathway for the patient to experience a cardiovascular event within the time period, and/or the most probable progression pathway for the patient to complete the time period without experiencing either of the aforementioned outcomes. “Progression” means “patient transitioning from lower or earlier CKD stage to higher or late CKD stage”.

The output can be outputted, e.g., displayed on a display, printed using a printing device, stored in a data memory, and/or transmitted to a separate computer system.

In addition to the output or instead of the output, information derived from the output can also be output.

For example, it is possible that the output is or contains a probability for the occurrence of a certain CKD result within the time period. The probability can be compared with a predefined threshold value. The threshold value can be determined by a physician, for example. For example, if the probability is less than the predefined threshold value, information may be outputted indicating that the patient's risk of developing the CKD outcome within the time period is low or less than the predefined threshold. For example, if the probability is greater than or equal to the predefined threshold value, information indicating that the patient is at risk (e.g., significant risk and/or non-negligible risk) of developing the CKD outcome within the time period may be provided.

The output and/or information derived therefrom can be issued to a physician and/or to the patient. The physician can see from the output and/or information derived therefrom how the course of the disease might develop within the time period. The physician may take action (e.g., plan and/or implement treatment, prescribe one or more medications, educate the patient, make recommendations) to mitigate or prevent the predicted disease course (e.g., when the predicted course would be a worsening of the patient's health). The patient can see how his/her disease state will likely develop. The patient can take measures to mitigate or prevent the predicted course of the disease, for example, by changing his/her lifestyle (smoking less, reducing weight, increasing physical activity, changing diet), and/or by complying with the measures provided by the physician.

In a preferred embodiment, those parameters that had the highest relevance to one or more predicted outcomes are identified.

Preferably, the determination of the highest relevance is made at least for the outcomes with the highest probability. The determination of the parameters with the highest relevance can be done by analyzing the model parameters of the trained machine learning model (see, e.g.: G. Montavon et al.: Layer-wise Relevance Propagation: An Overview, in: Explainable AI: Interpreting, Explaining and Visualizing Deep Learning (pp. 193-209), DOI: 10.1007/978-3-030-28954-6_10). The model parameters indicate which parameters were extracted from the different embeddings and/or which parameters were weighted with a higher weight during training. The determined parameters with the highest relevance (e.g., the top 3 or top 5 or top 10 or any other number) can be outputted together with the output and/or outcomes they refer to. The determined parameters with the highest relevance can be regarded as risk factors for the development of the respective outcome.

Based on the parameters determined, the physician can recognize, for example, which parameters have an influence on a negative outcome and can adjust the therapy plan accordingly. If, for example, elevated blood pressure is the parameter that has the greatest influence on the outcome “CV event”, the physician can take measures to lower the blood pressure.

It is also possible that measures (recommendations) are issued to the physician that can lead to a reduction in the probability of the CKD outcome occurring.

In a preferred embodiment, the new patient (i.e., the patient for whom a prediction is made) is placed in a group of similar patients.

Similarity can be determined in terms of age, gender, ethnicity, body mass index, current CKD status, previous disease course, and/or other patient data and/or a combination of patient data.

Similarity can be determined based on necessary conditions and/or proximity to one or more parameters. It may be, for example, that in a first step such (reference) patients are sorted out who do not fulfill one or more conditions. One such condition may be age or membership in an age group. For example, the (reference) patients can be divided into age groups, e.g., under 20 years, 21 to 30 years, 31 to 40 years, 42 to 50 years, 51 to 60 years, 61 to 70 years, 71 to 80 years, over 80 years. For the new patient, the age and the age group in which the patient is located are determined (e.g., based on the patient data). For further similarity analysis, only those (reference) patients are considered who are in the same age group as the new patient. The same procedure can be followed with other/further patient data, such as, e.g., current CKD stage, sex, ethnicity.

Alternatively, or in addition to the necessary conditions, a similarity or distance measure can be determined. For each reference patient of a multitude of reference patients as well as for the new patient, one or more patient data can be combined (and/or aggregated) in a feature vector. A similarity measure or a distance measure between the feature vector of the new patient and the feature vectors of the reference patients can be calculated. Examples of similarity or distance measures are cosine similarity, Manhattan distance, Euclidean distance, Minkowski distance, and/or other measures and/or a combination of different measures.

Those (reference) patients can be considered similar to a new patient where the similarity measure is above a defined threshold and/or the distance measure is below a defined threshold. The threshold value may be defined, for example, by a physician. It is also possible that the threshold is variable, so that a physician can change the threshold if too few similar (reference) patients are identified.

Once one or more similar (reference) patient(s) have been identified, the disease history of these (reference) patient(s), e.g., the CKD outcome within the defined time period, can be outputted. A physician and/or the new patient can then see what disease course similar (reference) patients had. Measures can be identified that were successfully taken in similar (reference) patients to reduce or prevent a worsening of the disease course. These measures can then also be performed on the new patient.

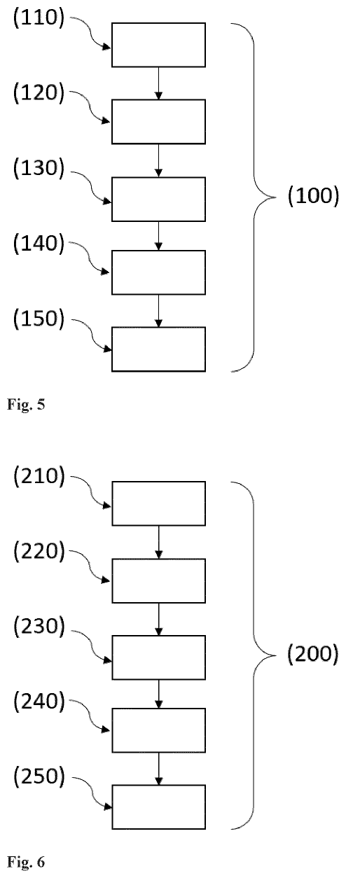

FIG. 5 shows schematically an embodiment of the computer-implemented prediction method of the present disclosure in the form of a flowchart.

The prediction method (100) comprises the steps:
(110) receiving patient data, wherein the patient data characterizes a patient suffering from chronic kidney disease (CKD) and being in one of a number of CKD stages of increasing severity,
(120) generating a graph based on the patient data, the graph comprising a patient vertex, a number of stage vertices, and a number of outcome vertices,
   wherein the patient vertex represents the patient,
   wherein each stage vertex represents one of the numbers of CKD stages,
   wherein each outcome vertex represents one outcome of chronic kidney disease within a time period,
   wherein the patient vertex is connected via an edge to the stage vertex representing the CKD stage the patient is in,
   wherein each stage vertex is connected via edges to all stage vertices representing a CKD stage of greater severity, and
   wherein each stage vertex is connected to each of the outcome stages via an edge,
(130) inputting the graph into a trained graph convolutional neural network, wherein the graph convolutional neural network was trained on training data to classify reference patients into the CKD outcomes, wherein the training data comprised, for each reference patient of a plurality of reference patients, i) a reference graph representing the reference patient and his/her chronic kidney disease course as input data, and ii) a CKD outcome as target data,
(140) receiving an output from the trained graph convolutional neural network, wherein the output indicates for one or more of the CKD outcomes a probability that CKD will reach the outcome in the patient within the time period,
(150) outputting and/or storing the output and/or information derived from the output, and/or transmitting the output and/or information derived from the output to a separate computer system.

FIG. 6 shows schematically an embodiment of the computer-implemented training method of the present disclosure in the form of a flowchart.

The training method (200) comprises the steps:
(210) providing training data, wherein the training data comprises, for each reference patient of a plurality of reference CKD patients, i) a refence graph as input data, and ii) a CKD outcome as target data, wherein each reference graph of each reference patient comprises:
   a patient vertex representing the reference patient,
   a stage vertex representing a baseline CKD stage of the reference patient and/or a stage vertex representing a destination CKD stage of the reference patient,
   an outcome vertex representing the CKD outcome that the reference patient developed during his/her chronic kidney disease course,
(220) inputting the reference graph into a graph convolutional neural network, wherein the graph convolutional neural network is configured to output a predicted CKD outcome based on the reference graph,
(230) receiving a predicted CKD outcome as an output of the graph convolutional neural network,
(250) quantifying a deviation between the predicted CKD outcome and the CKD outcome that the reference patient developed during his/her chronic kidney disease course,
(240) reducing the deviation by modifying parameters of the graph convolutional neural network.

The operations in accordance with the teachings herein may be performed by at least one computer system specially constructed for the desired purposes or general-purpose computer system specially configured for the desired purpose by at least one computer program stored in a typically non-transitory computer readable storage medium.

A “computer system” is a system for electronic data processing that processes data by means of programmable calculation rules. Such a system usually comprises a “computer”, that unit which comprises a processor for carrying out logical operations, and also peripherals.

In computer technology, “peripherals” refer to all devices which are connected to the computer and serve for the control of the computer and/or as input and output devices. Examples thereof are monitor (screen), printer, scanner, mouse, keyboard, drives, camera, microphone, loudspeaker, etc. Internal ports and expansion cards are, too, considered to be peripherals in computer technology.

Computer systems of today are frequently divided into desktop PCs, portable PCs, laptops, notebooks, netbooks and tablet PCs and so-called handhelds (e.g., smartphone); all these systems can be utilized for carrying out the invention.

The term “non-transitory” is used herein to exclude transitory, propagating signals or waves, but to otherwise include any volatile or non-volatile computer memory technology suitable to the application.

The term “computer” should be broadly construed to cover any kind of electronic device with data processing capabilities, including, by way of non-limiting example, personal computers, servers, embedded cores, computing system, communication devices, processors (e.g., digital signal processor (DSP)), microcontrollers, field programmable gate array (FPGA), application specific integrated circuit (ASIC), etc.) and other electronic computing devices.

The term “process” as used above is intended to include any type of computation or manipulation or transformation of data represented as physical, e.g., electronic phenomena which may occur or reside e.g., within registers and/or memories of at least one computer or processor. The term processor includes a single processing unit or a plurality of distributed or remote such units.

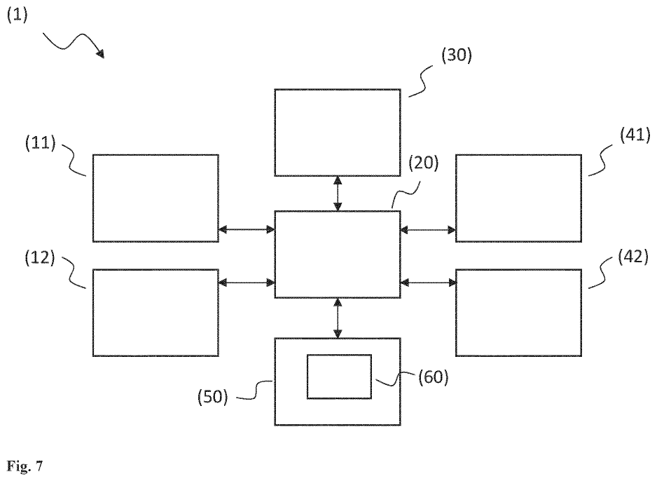

FIG. 7 illustrates a computer system (1) according to some example implementations of the present disclosure in more detail.

Generally, a computer system of exemplary implementations of the present disclosure may be referred to as a computer and may comprise, include, or be embodied in one or more fixed or portable electronic devices. The computer may include one or more of each of a number of components such as, for example, a processing unit (20) connected to a memory (50) (e.g., storage device).

The processing unit (20) may be composed of one or more processors alone or in combination with one or more memories. The processing unit (20) is generally any piece of computer hardware that is capable of processing information such as, for example, data, computer programs and/or other suitable electronic information. The processing unit (20) is composed of a collection of electronic circuits some of which may be packaged as an integrated circuit or multiple interconnected integrated circuits (an integrated circuit at times more commonly referred to as a “chip”). The processing unit (20) may be configured to execute computer programs, which may be stored onboard the processing unit (20) or otherwise stored in the memory (50) of the same or another computer.

The processing unit (20) may be a number of processors, a multi-core processor or some other type of processor, depending on the particular implementation. For example, it may be a central processing unit (CPU), a field programmable gate array (FPGA), a graphics processing unit (GPU) and/or a tensor processing unit (TPU). Further, the processing unit (20) may be implemented using a number of heterogeneous processor systems in which a main processor is present with one or more secondary processors on a single chip. As another illustrative example, the processing unit (20) may be a symmetric multi-processor system containing multiple processors of the same type. In yet another example, the processing unit (20) may be embodied as or otherwise include one or more ASICs, FPGAs or the like. Thus, although the processing unit (20) may be capable of executing a computer program to perform one or more functions, the processing unit (20) of various examples may be capable of performing one or more functions without the aid of a computer program. In either instance, the processing unit (20) may be appropriately programmed to perform functions or operations according to example implementations of the present disclosure.

The memory (50) is generally any piece of computer hardware that can store information such as, for example, data, computer programs (e.g., computer-readable program code (60)) and/or other suitable information either on a temporary basis and/or a permanent basis. The memory (50) may include volatile and/or non-volatile memory, and may be fixed or removable. Examples of suitable memory include random access memory (RAM), read-only memory (ROM), a hard drive, a flash memory, a thumb drive, a removable computer diskette, an optical disk, a magnetic tape or some combination of the above. Optical disks may include compact disks-read only memory (CD-ROM), compact disk-read/write (CD-R/W), DVD, Blu-ray disk or the like. In various instances, the memory may be referred to as a computer-readable storage medium or data memory. The computer-readable storage medium is a non-transitory device capable of storing information, and is distinguishable from computer-readable transmission media such as electronic transitory signals capable of carrying information from one location to another. Computer-readable medium as described herein may generally refer to a computer-readable storage medium or computer-readable transmission medium.

In addition to the memory (50), the processing unit (20) may also be connected to one or more interfaces for displaying, transmitting and/or receiving information. The interfaces may include one or more communications interfaces and/or one or more user interfaces. The communications interface(s) may be configured to transmit and/or receive information, such as to and/or from other computer(s), network(s), database(s) or the like. The communications interface may be configured to transmit and/or receive information by physical (wired) and/or wireless communications links. The communications interface(s) may include interface(s) (41) to connect to a network, such as using technologies such as cellular telephone, Wi-Fi, satellite, cable, digital subscriber line (DSL), fiber optics and the like. In some examples, the communications interface(s) may include one or more short-range communications interfaces (42) configured to connect devices using short-range communications technologies such as NFC, RFID, Bluetooth, Bluetooth LE, ZigBee, infrared (e.g., IrDA) or the like.

The user interfaces may include a display (30). The display (screen) may be configured to present or otherwise display information to a user, suitable examples of which include a liquid crystal display (LCD), light-emitting diode display (LED), plasma display panel (PDP) or the like. The user input interface(s) (11) may be wired or wireless, and may be configured to receive information from a user into the computer system (1), such as for processing, storage and/or display. Suitable examples of user input interfaces include a microphone, image or video capture device, keyboard or keypad, joystick, touch-sensitive surface (separate from or integrated into a touchscreen) or the like. In some examples, the user interfaces may include automatic identification and data capture (AIDC) technology (12) for machine-readable information. This may include barcode, radio frequency identification (RFID), magnetic stripes, optical character recognition (OCR), integrated circuit card (ICC), and the like. The user interfaces may further include one or more interfaces for communicating with peripherals such as printers and the like.

As indicated above, program code instructions (60) may be stored in memory (50), and executed by processing unit (20) that is thereby programmed, to implement functions of the systems, subsystems, tools and their respective elements described herein. As will be appreciated, any suitable program code instructions (60) may be loaded onto a computer or other programmable apparatus from a computer-readable storage medium to produce a particular machine, such that the particular machine becomes a means for implementing the functions specified herein. These program code instructions (60) may also be stored in a computer-readable storage medium that can direct a computer, processing unit or other programmable apparatus to function in a particular manner to thereby generate a particular machine or particular article of manufacture. The instructions stored in the computer-readable storage medium may produce an article of manufacture, where the article of manufacture becomes a means for implementing functions described herein. The program code instructions (60) may be retrieved from a computer-readable storage medium and loaded into a computer, processing unit or other programmable apparatus to configure the computer, processing unit or other programmable apparatus to execute operations to be performed on or by the computer, processing unit or other programmable apparatus.

Retrieval, loading and execution of the program code instructions (60) may be performed sequentially such that one instruction is retrieved, loaded and executed at a time. In some example implementations, retrieval, loading and/or execution may be performed in parallel such that multiple instructions are retrieved, loaded, and/or executed together. Execution of the program code instructions (60) may produce a computer-implemented process such that the instructions executed by the computer, processing circuitry or other programmable apparatus provide operations for implementing functions described herein.

Execution of instructions by processing unit, or storage of instructions in a computer-readable storage medium, supports combinations of operations for performing the specified functions. In this manner, a computer system (1) may include processing unit (20) and a computer-readable storage medium or memory (50) coupled to the processing circuitry, where the processing circuitry is configured to execute computer-readable program code instructions (60) stored in the memory (50). It will also be understood that one or more functions, and combinations of functions, may be implemented by special purpose hardware-based computer systems and/or processing circuitry which perform the specified functions, or combinations of special purpose hardware and program code instructions.

Further embodiments of the present disclosure include:
1. A computer-implemented method comprising:
   receiving patient data, wherein the patient data characterizes a patient suffering from chronic kidney disease (CKD) and being in one of a number of CKD stages of increasing severity,
   generating a graph based on the patient data, the graph comprising a patient vertex, a number of stage vertices, and a number of outcome vertices,
      wherein the patient vertex represents the patient,
      wherein each stage vertex represents one of the number of CKD stages,
      wherein each outcome vertex represents one outcome of chronic kidney disease within a time period,
      wherein the patient vertex is connected via an edge to the stage vertex representing the CKD stage the patient is in,
      wherein each stage vertex is connected via edges to all stage vertices representing a CKD stage of greater severity, and
      wherein each stage vertex is connected to each of the outcome stages via an edge,
   inputting the graph into a trained graph convolutional neural network, wherein the graph convolutional neural network was trained on training data to classify reference patients into the CKD outcomes, wherein the training data comprised, for each reference patient of a plurality of reference patients, i) a graph as input data, and ii) a CKD outcome as target data,
   receiving an output from the trained graph convolutional neural network, wherein the output indicates for one or more of the CKD outcomes a probability that CKD will reach the outcome in the patient within the time period,
   outputting the output and/or storing the output and/or transmitting the output to a separate computer system.
2. The method of embodiment 1,
   wherein the patient vertex is connected via a directed edge to the stage vertex representing the CKD stage the patient is in,
   wherein each stage vertex is connected via directed edges to all stage vertices representing a CKD stage of greater severity,
   wherein each stage vertex is connected to each of the outcome stages via a directed edge.
3. The method of embodiment 1 or 2, wherein at least a portion of the CKD stages are determined based on a glomerular filtration rate or an estimated glomerular filtration rate.
4. The method of any one of embodiments 1 to 3, wherein the CKD outcomes comprise one or more of the following outcomes: a cardiovascular event, a major adverse cardiovascular event, end-stage renal disease, death, no outcome.
5. The method of embodiment 4, wherein the major adverse cardiovascular event is myocardial infarction, unstable angina, need for revascularization, heart failure and/or stroke.
6. The method of any one of embodiment s 1 to 5, wherein the time period is 1, 2, 3, 4, 5, 6, 7, 8, 9, or 10 years.
7. The method of any one of embodiments 1 to 6, wherein the patient vertex comprises a patient embedding, the patient embedding representing the patient, the patient embedding being generated based on patient data.
8. The method of embodiment 7, wherein the patient data comprises one or more of the following: age; sex; ethnicity; current CKD stage (e.g., according to the NKF staging system); presence of one or more of the following conditions and/or information about how long the one or more conditions have been present: hypertension, diabetes, cardiovascular disease(s); GFR; eGFR; uACR; LDL; corrected calcium; systolic blood pressure; body mass index; information on whether the patient smokes and/or what quantities the patient consumes.
9. The method of any one of embodiments 1 to 8, wherein weights are assigned to the edges between each two stage vertices, the weights reflecting the percentage of reference patients who during their disease course had a baseline CKD stage corresponding to one of the two stage vertices and had a destination CKD stage corresponding to the other of the two stage vertices.
10. The method of any one of embodiments 1 to 9, wherein weights are assigned to edges between each pair consisting of a stage vertex and an outcome vertex, the weights reflecting the percentage of reference patients diagnosed with a baseline CKD stage corresponding to the stage vertex, and who had an outcome corresponding to the outcome vertex during their disease course.
11. The method of any one of embodiments 1 to 10, wherein the machine learning model is a graph convolutional neural network.
12. The method of any one of embodiments 1 to 11, comprising:
   determining a CDK outcome which has the highest probability,
   outputting the CDK output with the highest probability.
13. The method of any one of embodiments 1 to 12, comprising:
   outputting the most probable progression pathway for the patient to develop end-stage renal disease within the time period, and/or the most probable progression pathway for the patient to experience a cardiovascular event within the time period, and/or the most probable progression pathway for the patient to complete the time period without experiencing either of the aforementioned outcomes.
14. The method of any one of embodiments 1 to 13, comprising:
   determining a number of parameters within patient data representing the patient that have the highest relevance to the one or more CKD outcomes,
   outputting the parameters.
15. The method of any one of embodiments 1 to 14, comprising:
   determining a number of reference patients having a defined similarity to the patient,
   outputting a disease history for one or more reference patients of the number of reference patients.
16. The method of any one of embodiments 1 to 15, wherein training of the graph convolutional neural network comprises:
   providing the training data, wherein each graph of each reference patient comprises:
      a patient vertex representing the reference patient
      a stage vertex representing a baseline CKD stage of the reference patient and/or a stage vertex representing a destination CKD stage of the reference patient,
      an outcome vertex representing the CKD outcome that the reference patient developed during his/her disease course,
   inputting the graph into the graph convolutional neural network,
   receiving as an output of the graph convolutional neural network, a predicted CKD outcome,
   quantifying a deviation between the predicted CKD outcome and the CKD outcome that the reference patient developed during his/her disease course,
   reducing the deviation by modifying parameters of the graph convolutional neural network.
17. A computer system comprising:
a processor; and
a memory storing an application program configured to perform, when executed by the processor, an operation, the operation comprising:
   receiving patient data, wherein the patient data characterizes a patient suffering from chronic kidney disease (CKD) and being in one of a number of CKD stages of increasing severity,
   generating a graph based on the patient data, the graph comprising a patient vertex, a number of stage vertices, and a number of outcome vertices,
      wherein the patient vertex represents the patient,
      wherein each stage vertex represents one of the numbers of CKD stages,
      wherein each outcome vertex represents one outcome of chronic kidney disease within a time period,
      wherein the patient vertex is connected via an edge to the stage vertex representing the CKD stage the patient is in,
      wherein each stage vertex is connected via edges to all stage vertices representing a CKD stage of greater severity, and
      wherein each stage vertex is connected to each of the outcome stages via an edge,
   inputting the graph into a trained graph convolutional neural network, wherein the graph convolutional neural network was trained on training data to classify reference patients into the CKD outcomes, wherein the training data comprised, for each reference patient of a plurality of reference patients, i) a graph as input data, and ii) a CKD outcome as target data,
   receiving an output from the trained graph convolutional neural network, wherein the output indicates for one or more of the CKD outcomes a probability that CKD will reach the outcome in the patient within the time period,
   outputting the output and/or storing the output and/or transmitting the output to a separate computer system.
18. A non-transitory computer readable storage medium having stored thereon software instructions that, when executed by a processor of a computer system, cause the computer system to execute the following steps:
   receiving patient data, wherein the patient data characterizes a patient suffering from chronic kidney disease (CKD) and being in one of a number of CKD stages of increasing severity,
   generating a graph based on the patient data, the graph comprising a patient vertex, a number of stage vertices, and a number of outcome vertices,
      wherein the patient vertex represents the patient,
      wherein each stage vertex represents one of the numbers of CKD stages,
      wherein each outcome vertex represents one outcome of chronic kidney disease within a time period,
      wherein the patient vertex is connected via an edge to the stage vertex representing the CKD stage the patient is in,
      wherein each stage vertex is connected via edges to all stage vertices representing a CKD stage of greater severity, and
      wherein each stage vertex is connected to each of the outcome stages via an edge,
   inputting the graph into a trained graph convolutional neural network, wherein the graph convolutional neural network was trained on training data to classify reference patients into the CKD outcomes, wherein the training data comprised, for each reference patient of a plurality of reference patients, i) a graph as input data, and ii) a CKD outcome as target data,
   receiving an output from the trained graph convolutional neural network, wherein the output indicates for one or more of the CKD outcomes a probability that CKD will reach the outcome in the patient within the time period,
   outputting the output and/or storing the output and/or transmitting the output to a separate computer system.

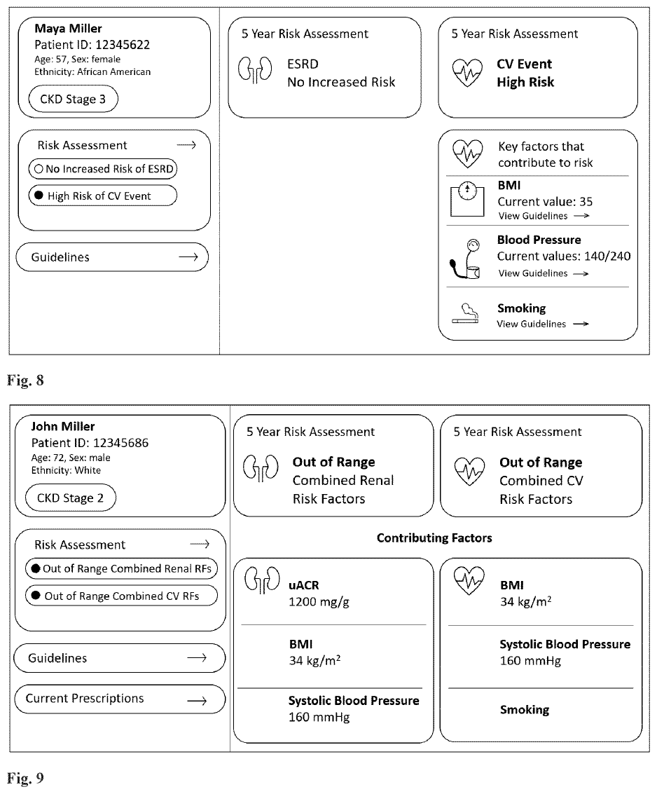

FIG. 8 shows an exemplary and schematic output of the computer system of the present disclosure. The output is a result of a prediction of a new patient's CKD progression generated with the trained machine learning model. Output is via a graphical user interface.

The new patient's name is Maya Miller. She has been assigned the patient ID 12345622. She is 57 years old, female and African American. At the time of prediction, she is in CKD stage 3. The patient data provided (age, sex, ethnicity, current CKD stage) and/or other/additional patient data may have been used for prediction.

The prediction showed no increased risk for the occurrence of the CKD outcome “ESRD” within the next 5 years. The prediction showed a high risk for the occurrence of a “CV event” within the next 5 years.

It is possible that the probability of the occurrence of the CKD outcome “ESRD” and the probability of the occurrence of the CKD outcome “CV event” within a period of 5 years were determined in the prediction and compared with a predefined threshold value in each case. It is possible that the probability of the CKD outcome “ESRD” occurring within the next 5 years is less than the respective predefined threshold value and that the probability of the CKD outcome “CV event” occurring within the next 5 years is greater than the respective predefined threshold value.

The key factors (risk factors) leading to the predicted outcome are indicated for the high risk of a cardiovascular event occurring. In this case, these were the high body mass index of 35, the high blood pressure of 140/240 and the fact that Maya Miller is a smoker.

The risk factors mentioned and identified by the computer system (BMI, blood pressure, smoker) are patient data that have been included in the prediction.

For the risk factors, a user (e.g., a physician) can use the arrows (View Guidelines) to display information on how the risk factors and hence the risk of a CV event within the next 5 years can be reduced.

FIG. 9 shows another exemplary and schematic output of the computer system of the present disclosure. The output is a result of a prediction of a new patient's CKD progression generated with the trained machine learning model. Output is via a graphical user interface.

The new patient's name is John Miller. He has been assigned the patient ID 12345686. He is 72 years old, male and white. At the time of prediction, he is in CKD stage 2. The patient data provided (age, sex, ethnicity, current CKD stage) and/or other/additional patient data may have been used for prediction.

In this example, patient data such as age, sex, ethnicity, current CKD stage, uACR, BMI and information on whether the patient is a smoker are used to calculate the probabilities of the patient reaching CKD outcome “ESRD” and a “CV event” occurring within 5 years. The probabilities are compared to predefined threshold values. In this example, both probabilities are above the predefined threshold values. This means that the risk for the CKD outcome “ESRD” and the occurrence of a “CV event” within the next 5 years is significant (not negligible). The term “Out of Range” reflects this result. The relevant parameters that led to the predicted outcome (contributing factors) are indicated for the corresponding CKD outcomes. In the case of the CKD outcome “ESRD”, these are the uACR value of 1200 mg/g, the BMI value of 34 kg/m² and the systolic blood pressure of 160 mmHg. In the case of the “CV event”, these are the BMI value of 34 kg/m², the systolic blood pressure of 160 mmHg and the fact that the patient is a smoker.

### Example

To ensure the accuracy and relevance of the training data, we manually constructed the training dataset based on data provided by research studies developed internally. The dataset has been in turn cross-validated against statistical distributions available in medical literature. We chose statistical distributions that are relevant to the problem that the machine learning model is intended to solve, i.e., CKD prediction method by stage of CKD.

The features in the dataset have been engineered using various statistical means, including averages, maximums, minimums, medians, and other measures. These features have also been defined based on their relevance to the problem that the machine learning model is designed to solve, thus aggregating on time windows, lags, etc.

To ensure that the training dataset is representative of the CKD population, we randomly selected the data points from the datasets compiled by our internal research on CKD. We used Bernoulli sampling, that is a random sampling technique used to select a subset of items from a larger population. In Bernoulli sampling, each item in the population is either included or excluded from the sample with a fixed probability p.

The basic steps of Bernoulli sampling are as follows:
1. Assign a fixed probability p to each item in the population.
2. Generate a random number between 0 and 1 for each item in the population.
3. Include the item in the sample if the random number is less than or equal to p. Exclude the item if the random number is greater than p.
4. Repeat steps 2 and 3 for each item in the population until the desired sample size is reached.

For example, suppose we want to select a sample of 100 items from a population of 1000 items. We can use Bernoulli sampling with a probability of 0.1 (p=0.1) to randomly select the sample. We generate a random number between 0 and 1 for each item in the population and include the item in the sample if the random number is less than or equal to 0.1. We repeat this process for each item in the population until we have selected 100 items for the sample.

This technique allowed us to select data points that are representative of the entire CKD population while ensuring that the distribution of the data points is consistent with the distribution of the population.

The resulting dataset consists of 10000 data points that however represent real patients in the population. To protect their privacy and anonymity, we constructed a synthetized dataset. Synthetic data is created by generating new data that has similar statistical properties to the original data but does not contain any actual data points from the original dataset. This means that the synthetic data cannot be used to directly identify individuals in the original dataset, thus protecting the privacy of individuals in the original dataset while still providing access to data that can be used for analysis and modeling.

There are several techniques for generating synthetic data, including generative models such as GANs (Generative Adversarial Networks), CTGAN (Conditional Tabular GAN), and VAEs (Variational Autoencoders). We generated synthetic data using CTGAN because it is designed specifically for tabular data, which is data that is organized into rows and columns.

Here is a list of steps that CTGAN typically follows to generate synthetic data:
1. Data preprocessing: The original dataset is preprocessed to remove and/or impute any missing values or invalid data points and transformed to a denormalized format that can be processed by the CTGAN model.
2. Model training: The CTGAN model is trained on the preprocessed original dataset, using a loss function that measures the difference between the statistical properties of the original dataset and the synthetic data generated by the model.
3. Sampling: Once the CTGAN model has been trained, it can be used to generate new synthetic data by sampling from the learned distribution. When generating synthetic data, CTGAN considers any conditioning variables provided by the user. For example, if the user specifies that the synthetic data should have the same distribution as the original dataset but with a specific value for a particular variable, CTGAN will generate synthetic data that meets this requirement.
4. Evaluation: The quality of the synthetic data generated by CTGAN is typically evaluated using various metrics, such as the Kolmogorov-Smirnov test or the Jensen-Shannon divergence, which measure the difference between the statistical properties of the synthetic data and the original dataset. If the synthetic data is found to be sufficiently similar to the original dataset, it can be used for downstream analysis or modeling.

Here is an example of how CTGAN can be used to generate synthetic data for a dataset representing CKD patients:
1. Data preprocessing: Suppose we have the original dataset that contains information about a patient that has been diagnosed with CKD stage 2 and CKD stage 3 over their history, with same variables tracked for each stage. We would first preprocess the data by imputing any missing values and converting the columns to have one row per patient, according to the following logic:
   a. Columns representing static values across stages are reported as is for each patient, e.g., “ETHNICITY”. Suppose the value for the above patient is “Afro-American” for the rows representing CKD stage 2 and stage 3, the result is a single patient row with “Afro-American” assigned to the “ETHNICITY” column.
   b. Columns representing values that vary across stages are pivoted into a composite column of stage and variable, e.g., “STAGE” and “EGFR” columns are denormalized into “STAGE2 EGFR” and “STAGE3_EGFR” columns, where the value is assigned by looking up the “EGFR” value from the row with the corresponding stage in the original dataset.
2. Model training: We then train the CTGAN model on the preprocessed dataset, using a loss function that measures the difference between the statistical properties of the original dataset and the synthetic data generated by the model.
3. Sampling: Once the CTGAN model has been trained, we can use it to generate new synthetic data. For example, we might ask CTGAN to generate 10,000 new data points that have the same statistical properties as the original dataset but with a specific value for the “EGFR” variable (such as “abnormal value”). CTGAN will then generate synthetic data that meets this requirement, while also preserving the statistical relationships between the other variables in the dataset.
4. Evaluation: We evaluate the quality of the synthetic data generated by CTGAN using various metrics, such as the Kolmogorov-Smirnov test or the Jensen-Shannon divergence. If the synthetic data is found to be sufficiently similar to the original dataset, it can be used for downstream analysis or modeling.

The resulting dataset is then pivoted back to the original data model, thus defined again by a composite primary key (“PATIENT_ID” and “STAGE”) and including both continuous and discrete features reorganized according to CKD stage, as depicted in the following table:

| Feature | Type | Description/possible values |
|---|---|---|
| PATIENT_ID | Primary key | De-identified ID, auto-increasing from 0 |
| STAGE | Primary key | Stage 1/Stage 2/Stage 3/Stage 4/Stage 5 |
| GENDER | Static | Male/female/unknown |
| IS DECEASED | Static | Yes/no |
| DEATH YEAR | Static | Only for deceased patients |
| ETHNICITY | Static | grouped in 5 years base |
| AGE | Stage-specific | Afro-American/Asian/Caucasian/ |
| | | Hispanic/unknown |
| CV EVENTS COUNT | Stage-specific | Count of CV events before the CKD stage |
| | | diagnosis date |
| HYPERTENSION | Stage-specific | Yes/no |
| DIABETES | Stage-specific | Yes/no |
| SMOKING | Stage-specific | Yes/no |
| CV PATIENT | Stage-specific | Yes/no |
| YEARS IN CKD | Stage-specific | Number of years as CKD patient |
| YEARS IN CV | Stage-specific | Number of years as CV patient |
| EGFR | Stage-specific | Average value of latest 3 months |
| UACR | Stage-specific | Average value of latest 3 months |
| LDL | Stage-specific | Average value of latest 3 months |
| SBP | Stage-specific | Average value of latest 3 months |
| CORRECTED | Stage-specific | Average value of latest 3 months |
| CALCIUM | | |
| BMI | Stage-specific | Average value of latest 3 months |

The synthetic training data were used to train a graph convolutional neural network as depicted in FIG. 4.

The graph convolutional neural network consisted of the following layers:
1. Input layer: This layer accepts the patient vertex and its properties matrix as input.
2. First GCN (Convolutional) layer: This layer reduces the dimensionality of the vertex's properties matrix from 15 dimensions to 10 dimensions. It is activated by the ReLu function and uses sum pooling.
3. Second GCN (Convolutional) layer: This layer reduces the dimensionality of the vertex's properties matrix from 10 to 6. It is activated by the ReLu function and uses max pooling.
4. Third GCN (Convolutional) layer: This layer reduces the dimensionality of the vertex's properties matrix from 6 to 4. It is activated by the ReLu function and uses max pooling.
5. First dense layer: This layer consists of 5 nodes and is activated with the ReLu function.
6. Second dense layer: This layer consists of 4 nodes and is activated with the ReLu function.
7. Output layer: This layer consists of 4 nodes and is activated with the sigmoid function with a threshold to perform the classification.

The model outputs can be categorized as follows:
Predictions: prediction is the highest level of dependent probabilities, which is reflecting the causalities & correlations between the input and the predicted output, as predictions the model has the following output:
   The major predicted outcome of the patient is whether the patient is at risk of ESRD, CV-event, both, or no outcome based on the input. To make a decision about this classification task, a threshold needs to be applied. The model has an auto-tuning tool to determine the threshold for a given cohort. Although death is one of the outcomes, it is not shown to the user. It is used to reduce bias and consider the effect of death on the training cohort, keeping the score and threshold consistent with the cohort statistics.
   Score that reflects the risk level: score is the probability calculated by the model for each outcome.
Probable progression pathways: Probable progression pathways are provided by the model depending on the predicted outcomes. The model gives independent probability-based output that explains the most repeated patient journeys towards the predicted outcome based on likelihood population. The pathways depend on the graph structure. These pathways include:
   Most repeated pathway toward CV-event.
   Most repeated pathway toward ESRD.
   Most repeated pathway toward safety “no-outcome”.

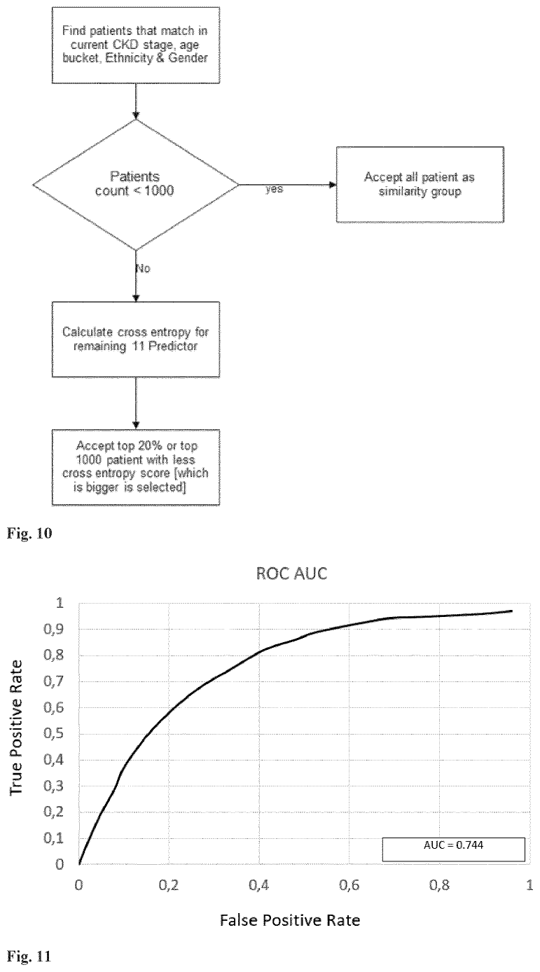

FIG. 10 explains how the model shortlists the cohort based on likelihood.
Interpretation is provided by the model to help the user understand the top risk factor that leads to the prediction. The model gives information on the top input data that lead to the risk score, along with an estimation of the contribution of that risk factor to the decision. Data-centric methodology is used for this purpose.
   The contribution of the risk factors is estimated as percentage.
   The model focuses on the contribution of the modifiable risk factors, the following list defined the modifiable risk factors from input data:
      SMOKING
      EGFR
      UACR
      LDL
      SBP
      CORRECTED CALCIUM
      BMI
   The contribution comes in 2 buckets: first for ESRD risk, and second for CV-event risk.

### Results:

The experiments show a high correlation between the selected input data and the outcomes. This reflects the model's ability to predict the risk of developing ESRD or CV-events for CKD patients in a 5-year time window, which can be trusted.

The evaluation matrix of the model includes the following:
Accuracy: the accuracy calculated as binary classification task, so the accuracy reflects the decision accuracy of (risky outcome, no risky outcome) made by the model.
Roc AUC: ROC AUC also was measured based on binary classification outcomes.
Sensitivity: Sensitivity has been measured based on the risky outcomes, so we have the sensitivity for CV-events, and the sensitivity for ESRD.
Specificity: Specificity has been measured based on the risky outcomes, so we have the Specificity for CV-events, and the specificity for ESRD.

The following table shows the experiments results:

| | Accuracy | ROC AUC | Sensitivity | Specificity |
|---|---|---|---|---|
| Binary Task | 0.77 | 0.75 | | |
| CV-event | | | 0.72 | 0.83 |
| ESRD | | | 0.81 | 0.77 |

FIG. 11 shows the corresponding ROC AUC curve.

### List of Acronyms

AIDC—automatic identification and data capture
ASIC—application specific integrated circuit
AUC—area under the curve
BMI—body mass index
CKD—chronic kidney disease
CKD—EPI-Chronic Kidney Disease Epidemiology Collaboration equation
CPU—central processing unit
CTGAN—Conditional Tabular Generative Adversarial Networks
CV—cardiovascular
DSL—digital subscriber line
DSP—digital signal processor
eGFR—estimated glomerular filtration rate
ERA—European Renal Association
ESRD—end-stage renal disease
FPGA—field programmable gate array
G—graph onto
GANs Generative Adversarial Networks
GFR—glomerular filtration rate
GCN-graph convolutional neural network
G^{T}—transformed graph
ICC—integrated circuit card
IL—input layer of graph convolutional neural network
IrDA—
KDIGO—Kidney Disease Improving Global Outcomes
LCD—liquid crystal display
LDL—low-density lipoprotein cholesterol
LED—light-emitting diode display
MACE—major adverse cardiovascular events
MDRD—Modification of Diet in Renal Disease
NFC—Near Field Communication
NKF—National Kidney Foundation in the United States
OCR—optical character recognition
OPL—operation layer
OUL—output layer
PC—personal computer
POM—pooling module
PRM—propagation module
PU—prediction unit
RAM—random access memory
RFID—radio frequency identification
ROC—Receiver Operating Characteristic
ROM—read-only memory
SBP—systolic blood pressure
SC—skip connection operation
SM—sampling modules SM
TPU—tensor processing unit
uACR—urine albumin-creatinine ratio
VAEs—Variational Autoencoders

What is claimed is:

1. A computer-implemented method comprising:
receiving patient data, wherein the patient data characterizes a patient suffering from chronic kidney disease (CKD) and being in one of a number of CKD stages of increasing severity, the number of CKD stages comprising: a cardiovascular event and end-stage renal disease;
generating a graph based on the patient data, the graph comprising a patient vertex, a number of stage vertices, and a number of outcome vertices;
wherein the patient vertex represents the patient;
wherein each stage vertex represents one of the number of CKD stages;
wherein each outcome vertex represents one outcome of chronic kidney disease within a time period;
wherein the patient vertex is connected via an edge to the stage vertex representing the CKD stage the patient is in;
wherein each stage vertex is connected via edges to all stage vertices representing a CKD stage of greater severity; and
wherein each stage vertex is connected to each of the outcome stages via an edge;
inputting the graph into a trained graph convolutional neural network, wherein the graph convolutional neural network was trained on training data to classify reference patients into the CKD outcomes, wherein the training data comprised, for each reference patient of a plurality of reference patients, i) a reference graph representing the reference patient and his/her chronic kidney disease course as input data, and ii) a CKD outcome of the reference patient as target data;
receiving an output from the trained graph convolutional neural network, wherein the output indicates for one or more of the CKD outcomes a probability that CKD will reach the outcome in the patient within the time period;
outputting and/or storing the output and/or information derived from the output, and/or transmitting the output and/or information derived from the output to a separate computer system.

2. The method of claim 1, wherein the patient vertex representing the patient is connected via a directed edge to the stage vertex representing the CKD stage the patient is in;
wherein each stage vertex representing one of the number of CKD stages is connected via directed edges to all stage vertices representing a CKD stage of greater severity;
wherein each stage vertex representing one of the number of CKD stages is connected to each of the outcome stages via a directed edge.

3. The method of claim 1, wherein each reference graph comprises:
a patient vertex representing the reference patient;
a stage vertex representing a baseline CKD stage of the reference patient and/or a stage vertex representing a destination CKD stage of the reference patient;
an outcome vertex representing the CKD outcome that the reference patient developed during his/her chronic kidney disease course.

4. The method of claim 1, wherein at least a portion of the CKD stages are determined based on a glomerular filtration rate or an estimated glomerular filtration rate.

5. The method of claim 1, wherein the CKD outcomes comprise one or more of the following outcomes: a cardiovascular event; a major adverse cardiovascular event; end-stage renal disease; death; an outcome other than a cardiovascular event, a major adverse cardiovascular event, end-stage renal disease, or death.

6. The method of claim 5, wherein the major adverse cardiovascular event is myocardial infarction, unstable angina, need for revascularization, heart failure and/or stroke.

7. The method of claim 1, wherein the CKD outcomes comprise: a cardiovascular event; end-stage renal disease; death; and an outcome other than a cardiovascular event, end-stage renal disease, or death.

8. The method of claim 1, wherein the CKD outcomes comprise: a cardiovascular event; end-stage renal disease.

9. The method of claim 1, wherein the CKD outcomes are: a cardiovascular event; end-stage renal disease; death; and an outcome other than a cardiovascular event, end-stage renal disease, or death.

10. The method of claim 1, wherein the time period is 1, 2, 3, 4, 5, 6, 7, 8, 9, or 10 years.

11. The method of claim 1, wherein the patient vertex comprises a patient embedding, the patient embedding representing the patient, the patient embedding being generated based on the patient data.

12. The method of claim 1, wherein the patient data comprises: a glomerular filtration rate and/or an albumin level.

13.-14. (canceled)

15. The method of claim 1, wherein the patient data comprises: age, sex, current CKD stage, GFR or eGFR, uACR, ethnicity, body mass index, LDL, systolic blood pressure, corrected calcium, information on whether the patient smokes, and/or information on whether the patient suffers from diabetes.

16. The method of claim 1, wherein weights are assigned to the edges between each two stage vertices of each reference graph, the weights reflecting the percentage of reference patients who during their disease course had a baseline CKD stage corresponding to one of the two stage vertices and had a destination CKD stage corresponding to the other of the two stage vertices.

17. The method of claim 1, wherein weights are assigned to edges between each pair consisting of a stage vertex and an outcome vertex of each reference graph, the weights reflecting the percentage of reference patients diagnosed with a baseline CKD stage corresponding to the stage vertex, and who had an outcome corresponding to the outcome vertex during their disease course.

18. The method of claim 1, comprising:
determining a CKD outcome which has the highest probability;
outputting the CKD output with the highest probability.

19. The method of claim 1, comprising:
determining and outputting the most probable course of the disease within the defined time period for the patient, from the CKD stage the patient is in, to the most probable CKD outcome for the patient.

20. The method of claim 1, comprising:
determining and outputting the most probable progression pathway for the patient to develop end-stage renal disease within the time period, and/or the most probable progression pathway for the patient to experience a cardiovascular event within the time period, and/or the most probable progression pathway for the patient to complete the time period without experiencing either of the aforementioned outcomes.

21. The method of claim 1, comprising:
determining a number of parameters within patient data representing the patient that have the highest relevance to the one or more CKD outcomes;
outputting the parameters.

22. The method of claim 1, comprising:
determining a number of reference patients having a defined similarity to the patient;
outputting a disease history for one or more reference patients of the number of reference patients.

23. The method of claim 1, wherein training of the graph convolutional neural network comprises:
providing the training data, wherein each reference graph of each reference patient comprises:
a patient vertex representing the reference patient;
a stage vertex representing a baseline CKD stage of the reference patient and/or a stage vertex representing a destination CKD stage of the reference patient;
an outcome vertex representing the CKD outcome that the reference patient developed during his/her chronic kidney disease course;
inputting the reference graph into the graph convolutional neural network;
receiving, as an output of the graph convolutional neural network, a predicted CKD outcome;
quantifying a deviation between the predicted CKD outcome and the CKD outcome that the reference patient developed during his/her chronic kidney disease course;
reducing the deviation by modifying parameters of the graph convolutional neural network.

24. A computer system comprising:
a processor; and
a memory storing an application program configured to perform, when executed by the processor, an operation, the operation comprising:
receiving patient data, wherein the patient data characterizes a patient suffering from chronic kidney disease (CKD) and being in one of a number of CKD stages of increasing severity, the number of CKD stages comprising: a cardiovascular event and end-stage renal disease;
generating a graph based on the patient data, the graph comprising a patient vertex, a number of stage vertices, and a number of outcome vertices;
wherein the patient vertex represents the patient;
wherein each stage vertex represents one of the numbers of CKD stages;
wherein each outcome vertex represents one outcome of chronic kidney disease within a time period;
wherein the patient vertex is connected via an edge to the stage vertex representing the CKD stage the patient is in;
wherein each stage vertex is connected via edges to all stage vertices representing a CKD stage of greater severity; and
wherein each stage vertex is connected to each of the outcome stages via an edge;
inputting the graph into a trained graph convolutional neural network, wherein the graph convolutional neural network was trained on training data to classify reference patients into the CKD outcomes, wherein the training data comprised, for each reference patient of a plurality of reference patients, (i) a reference graph representing the reference patient and his/her chronic kidney disease course as input data, and ii) a CKD outcome of the reference patient as target data;
receiving an output from the trained graph convolutional neural network, wherein the output indicates for one or more of the CKD outcomes a probability that CKD will reach the outcome in the patient within the time period;
outputting and/or storing the output and/or information derived from the output, and/or transmitting the output and/or information derived from the output to a separate computer system.

25. A non-transitory computer-readable storage medium having stored thereon software instructions that, when executed by a processor of a computer system, cause the computer system to execute the following steps:
receiving patient data, wherein the patient data characterizes a patient suffering from chronic kidney disease (CKD) and being in one of a number of CKD stages of increasing severity, the number of CKD stages comprising: a cardiovascular event and end-stage renal disease;
generating a graph based on the patient data, the graph comprising a patient vertex, a number of stage vertices, and a number of outcome vertices;
wherein the patient vertex represents the patient;
wherein each stage vertex represents one of the numbers of CKD stages;
wherein each outcome vertex represents one outcome of chronic kidney disease within a time period;
wherein the patient vertex is connected via an edge to the stage vertex representing the CKD stage the patient is in;
wherein each stage vertex is connected via edges to all stage vertices representing a CKD stage of greater severity; and
wherein each stage vertex is connected to each of the outcome stages via an edge;
inputting the graph into a trained graph convolutional neural network, wherein the graph convolutional neural network was trained on training data to classify reference patients into the CKD outcomes, wherein the training data comprised, for each reference patient of a plurality of reference patients, i) a reference graph representing the reference patient and his/her chronic kidney disease course as input data, and ii) a CKD outcome of the reference patient as target data;
receiving an output from the trained graph convolutional neural network, wherein the output indicates for one or more of the CKD outcomes a probability that CKD will reach the outcome in the patient within the time period;
outputting and/or storing the output and/or information derived from the output, and/or transmitting the output and/or information derived from the output to a separate computer system.
